(19) United States
(12) Patent Application Publication
(10) Pub. No.: US 2002/0072625 A1
(43) Pub. Date: Jun. 13, 2002

(54) Materials and methods for measuring chelate: anti-chelate binding by fluorescence polarization immunoassay

(76) Inventor: David Kenneth Johnson, Silver Lake, WI (US)

Correspondence Address: DEANNA J. NELSON, 104 TASMAN COURT, CARY, NC, 27513, US

(21) Appl. No.: 09/733,801

(22) Filed: Dec. 9, 2000

Related U.S. Application Data
(60) Provisional application No. 60/170,246, filed on Dec. 10, 1999.

(51) Int. Cl. G01N 33/53; G01N 33/537; G01N 33/543; C07F 15/00; C07F 7/30; C07F 7/28; C07F 5/06

(52) U.S. Cl. 556/56; 556/94; 556/137; 556/148; 556/183; 435/7.92

(57) ABSTRACT

The present invention relates to chelate-fluorophore conjugates and their use in monitoring binding reactions between a chelate and a macromolecular biological binding agent, such as an antibody. Such monitoring is used both to identify anti-chelate antibodies that have a desired metal selectivity and to configure immunoassays that provide a means of detecting and quantitating a given metal ion.

### CROSS-REFERENCE TO RELATED APPLICATIONS

This patent application is based on U.S. Provisional Patent Application Serial No. 60/170,246, filed Dec. 10, 1999, entitled “Materials and Methods for Measuring Chelate: Anti-Chelate Binding by Fluorescence Polarization Immunoassay” of David K. Johnson. This patent application is also related to co-pending U.S. patent application Ser. No. 09/148,733, filed Sep. 4, 1998, entitled: Novel Diethylenetriamine-N,N′,N″-Triacetic Acid Derivatives” of David K. Johnson.

### STATEMENT REGARDING FEDERALLY SPONSORED RESEARCH OR DEVELOPMENT

No Federally sponsored research or development was provided to this application.

### FIELD OF THE INVENTION

This invention relates to the field of immunoassay techniques for measuring the concentration of a given metal ion, using antibodies that bind a chelated form of that metal ion. In particular, it relates to chelate compositions that incorporate a fluorescent dye and to fluorescence-based methods for selecting and utilizing anti-chelate antibodies.

### BACKGROUND OF THE INVENTION

Ethylenediamine-N,N,N′,N′-tetraacetic acid (hereafter EDTA) and diethylenetriamine-N,N,N′,N″-N″-pentaacetic acid (hereafter DTPA) are synthetic chelating agents well known to the art that form stable complexes with a wide range of metal ions [Sillen and Martell (eds) Stability Constants of Metal Ion Complexes, Chemical Society, London (1964); Martell and Smith (eds) Critical Stability Constants, Plenum, New York (1974)]. Derivatives of EDTA and DTPA that contain a side chain comprising a p-aminobenzyl- or p-isothiocyanatobenzyl-group attached at a methylene carbon atom of the polyamine backbone have been disclosed by Sundberg et al. [J. Med. Chem., 17: 1304-7 (1974)] and Brechbiel et al. [Inorg. Chem., 25: 2772-81 (1986); Bioconjugate Chem., 2: 187-94 (1991)]. The side chain functionality may be used to covalently link the chelating agent to any other molecule, such as a protein [Meares et al., Anal. Biochem., 142: 68-78 (1984)] or, as in the case of the present invention, a reporter molecule.

Antibodies that bind selectively to the coordination complexes (hereafter termed chelates; a metal-binding agent that contains more than one metal-binding atom is termed a chelating agent or chelator; and the complex formed between a chelator and a metal ion is called a chelate) formed between a given metal ion and EDTA or DTPA have been disclosed by Meares et al. [U.S. Pat. No. 4,722,892 (1988)], Goodwin et al. [J. Nucl. Med., 29: 266-74 (1988)], Le Doussal et al. [Cancer Res., 50: 3445-52 (1989)] and Blake et al. [J. Biol. Chem., 271: 27677-85 (1996)]. The use of antibodies that selectively bind EDTA chelates to configure enzyme linked immunosorbent assays (hereafter ELISA) for measuring the concentration of a given metal ion has been disclosed by Chakrabarti et al. [Anal. Biochem., 217: 70-75 (1994)] and Khosraviani et al. [Environ. Sci. Technol., 32: 137-42 (1998)] for the metals indium(III) and cadmium(II), respectively.

Fluorescent dye molecules (hereafter termed fluorophores) that contain a side chain functionality that either (a) is an amine or (b) is reactive with an amine are available commercially (e.g., from Sigma-Aldrich, St. Louis, Mo. or Molecular Probes, Eugene, Oreg.). General methods for covalently linking such fluorophores to other molecules have been reviewed by Brinkley [Bioconjugate Chem., 3: 2-13 (1992)].

The use of fluorescence polarization as a technique for studying antibody-antigen interactions in solution was developed by Dandliker et al. [Immunochem., 10: 219-27 (1973)]. Fluorescence polarization immunoassay (hereafter FPIA) has subsequently been applied to the measurement of a variety of low molecular weight analytes such as drugs and hormones [Jolley, J. Anal. Toxicol., 5: 236-240 (1981)]. Fluorescence polarization assays for metal ions based on antibodies to metal ion-ligand complexes have been disclosed by Johnson [U.S. Pat. No. 5,631,172 (1997)].

### SUMMARY OF THE INVENTION

The present invention provides compositions comprising an EDTA or DTPA chelate covalently linked to a fluorophore, methods for the use of said compositions in screening and characterizing anti-chelate antibodies and immunoassays for metal ions based on said chelate compositions and antibodies.

Chelate compositions of the present invention have structure (A) (FIG. 1) and are obtained either by (a) combining an amine-reactive EDTA or DTPA derivative with a fluorophore bearing an amine side chain or by (b) combining an amine-reactive fluorophore derivative with an EDTA or DTPA derivative bearing a side chain amine, followed in either case by (c) complexation of the resulting conjugate with the metal ion of choice.

Metals that may be incorporated into the compositions of the present invention comprise bismuth, tin, lead, aluminum, gallium, indium, thallium or any of the elements of groups IIa, IIIa, IVa, Va, VIa, VIIa, VIII Ib or VIII IIb of the periodic table of the elements, any member of the lanthanide series of the elements or any member of the actinide series of the elements except lawrencium, as illustrated in FIG. 2.

The metal selectivity of the immune response in animals inoculated with an EDTA or DTPA chelate of a given metal (hereafter termed the target chelate) is characterized by combining an aliquot of material thought to contain an anti-chelate antibody (such as a serum sample or a hybridoma supernatant) with an aliquot of either a target or a non-target chelate composition of the present invention and measuring the polarization of the fluorescent signal obtained when the resulting solution is excited with plane polarized light. The production of antibodies that bind to the target chelate is characterized by a polarized emission when combined with a target chelate-fluorophore composition while the absence of a response to the target chelate is indicated by a low polarization in the presence of said composition. A metal-selective antibody is present if, at a fixed antibody concentration, a target chelate-fluorophore composition produces a polarized signal while the same concentration of a corresponding non-target chelate-fluorophore composition produces a signal of significantly lower polarization. Any given antibody preparation may be readily probed in this way for metal selectivity using as many different non-target chelate-fluorophore compositions as may be required by the particular analytical application envisioned for that antibody, namely, by the nature and concentrations of non-target metals expected to be present in the sample matrix of interest. As an antibody raised against an EDTA or DTPA chelate of any given metal is potentially cross-reactive with approximately 70 other metals, this ability to rapidly screen for non-target reactivity is a particular preferred embodiment of the present invention.

Fluorescence polarization screening using target and non-target chelate-fluorophore compositions of the present invention may be used to characterize and track the polyclonal antibody response in animals immunized with any EDTA or DTPA chelate. Such characterization may be used to identify particular polyclonal antisera for subsequent immunoassay use, when an assay is to be based on polyclonal antibodies, or may be used to identify the most promising animals and/or stage of the polyclonal response when planning fusions to produce monoclonal antibodies. Hybridoma supernatants can be readily screened by the same procedure to identify clones with desirable metal selectivities for use in monoclonal antibody-based immunoassays for metal ions. Alternative methods known in the art for screening the specificity of anti-hapten antibodies using fluorescence polarization (e.g., by adding a large excess of a non-target hapten to a solution containing a target hapten-fluorophore tracer bound to the antibody and monitoring displacement of the tracer), are prone to artifacts when the non-target haptens are metal-chelate complexes, because the latter produce a high concentration of electronic charge around the antibody molecule that can alter its binding properties.

The compositions of the present invention may be used to rapidly and readily define the pattern of reactivity, across any desired spectrum of metal ions, of an antibody raised against an EDTA- or DTPA-chelate of any particular target metal ion and thus, to select antibodies for subsequent use in an immunoassay. Such immunoassays may be conducted in any assay format suitable for the measurement of a low molecular weight hapten, such as the ELISA [Chakrabarti et al.; Khosraviani et al.] or radioimmunoassay [Ogan et al., J. Pharm. Sci., 82: 475-479 (1993)] formats that were used in prior art immunoassays based on anti-chelate antibodies. A preferred embodiment of the present invention uses a target chelate-fluorophore composition in a competitive binding FPIA format to provide a homogeneous immunoassay that detects the target metal ion with high sensitivity and specificity. FPIA tests of this type can detect lead in samples of municipal drinking water, airborne particulates and soil with sensitivities well below regulatory action levels for lead contamination in these matrices.

### BRIEF DESCRIPTION OF THE DRAWINGS

FIG. 1 discloses the general formula for a chelate-fluorophore conjugate of the present Invention.

FIG. 2 presents those elements of the Periodic Table of the Elements that form stable chelate complexes with EDTA and DTPA [after Reardan et al., Nature, 316, 265-268 (1985)].

FIG. 3 presents a particularly preferred group of precursors useful in the synthesis of chelate-fluorophore conjugates of the present Invention.

FIG. 4 presents methods for the synthesis of chelator-fluorophore conjugates of the present Invention.

FIG. 5 presents a fluorescence polarization titer curve wherein a fixed concentration of a target chelate-fluorophore conjugate of the present Invention comprising a lead chelate-fluorophore tracer is combined with serial dilutions of rabbit antiserum, and the resulting fluorescence polarization is determined.

FIG. 6 presents fluorescence polarization titer curves wherein the same fixed concentration of a target (lead) or either of two non-target (iron, aluminum) chelate-fluorophore tracers is combined with serial dilutions of rabbit antiserum, and the resulting fluorescence polarization is determined.

FIG. 7 presents titer curves wherein the same fixed concentration of a target (lead) or any one of four non-target (chromium, zinc, copper, nickel) chelate-fluorophore tracers is combined with serial dilutions of rabbit antiserum, and the resulting fluorescence polarization is determined.

FIG. 8 presents a standard curve obtained using an FPIA immunoassay of the present Invention for ionic lead(II) in 1.0 M nitric acid.

FIG. 9 depicts the linear regression analysis for 55 soil samples tested for lead content using an FPIA immunoassay of the present Invention or using atomic absorption spectroscopy (AAS).

FIG. 10 depicts a standard curve for an immunoassay of the present Invention for ionic lead(II) in a nitric acid+hydrochloric acid mixture used to extract airborne particulates from a filter.

FIG. 11 compares the experimental results obtained using an immunoassay of the present Invention with those obtained by atomic absorption spectroscopic analysis of 15 extracts of filters used to collect airborne particulates.

FIG. 12 presents the results of FPIA immunoassay of the present Invention for drinking water spiked with lead(II) at the concentrations shown.

FIG. 13 presents a standard curve obtained using an immunoassay of the present Invention for standards consisting of the pre-formed 1:1 Pb-EDTA chelate complex.

### DETAILED DESCRIPTION OF THE INVENTION

Antibodies produced by immunizing animals with chelates of EDTA or DTPA can show remarkable selectivity for the chelate formed with the immunizing metal compared to other metal complexes of the same chelating agent. The chelating agents themselves show minimal specificity, forming stable chelates with over 70 different metals. However, subtle differences in chelate “shape” from one metal to another (i.e., structural characteristics such as precise bond distances and bond angles, overall charge, charge distribution and charge:radius ratio, configurational differences, and open sites in the coordination sphere) apparently combine to allow an antibody binding site, which has an exquisite ability to differentiate between closely related molecular structures, to distinguish between different metal chelates formed from a single chelating agent.

Because of the large number of potential cross-reactivities, the screening of such anti-chelate antibodies and selection of an optimum antibody for any particular analytical application present a particular challenge. To date, most anti-chelate antibody development has been focused on monoclonal antibodies, and screening of the initial polyclonal response has been confined to testing for the presence of antibodies that bind the target chelate but not the free chelating agent. After fusions have been performed, screening of hybridoma supernatants for metal selectivity, to the extent that it has been performed at all, has been done by indirect ELISA techniques. In these methods the target chelate is immobilized on a suitable solid phase, such as a microtiter plate, and the ability of solution phase non-target chelates to inhibit binding of mouse immunoglobulin to the solid phase is evaluated. Such screening procedures are cumbersome and subject to artifacts, inasmuch as the competition is established between chelates in two different phases. The extent to which subtleties of chelate shape (on which antibody recognition is based) are modified when that chelate is conjugated to a carrier protein and physically adsorbed to a polymer surface is unknown. As a result of such limitations, definitive data on the metal selectivity of anti-chelate monoclonal antibodies have only been obtained ex post facto by equilibrium binding studies that measure dissociation constants in solution for an antibody, once selected, produced, and purified in quantities sufficient to perform such analyses. Partly as a result of such limitations, it has proven challenging to obtain anti-EDTA monoclonal antibodies that do not cross-react significantly with one or more non-target metal ions and that lead to immunoassays of adequate sensitivity to meet regulatory requirements. For example, Khosraviani et al. [Environ. Sci. Technol., 32: 137-42 (1998)] have disclosed an ELISA assay for cadmium that is based on a monoclonal anti-EDTA-Cd(II) antibody; this assay shows significant cross-reaction with mercury(II) and has a limit of detection for cadmium in water that exceeds regulatory action levels by an order of magnitude or more. Similarly, there are no well documented reports in the art of successfully measuring the concentration of a metal ion in a soil sample by anti-chelate immunoassay, other than a semi-quantitative ELISA for mercury in soil (EPA Method 4500 in draft SW-846, OSW, U.S.E.P.A.)

The present invention provides fluorescent chelate compositions that can be used in conjunction with a fluorescence polarization read-out to provide direct information about the metal selectivity of any anti-EDTA or anti-DTPA antibody in solution at equilibrium. Results can be obtained rapidly, and it is not necessary for the antibody to be in pure form, so the technique is applicable to serum samples, hybridoma supernatants and similar complex mixtures. Both target and non-target chelate-fluorophore compositions are used in such screening. In addition, the target chelate-fluorophore composition may be used in a preferred immunoassay format, namely, competitive binding FPIA, to measure the concentration of target metal present in a given sample. The FPIA can be used to measure a preferred target metal, lead(II), in complex sample matrices that include soil and airborne particulates. Only the target chelate-fluorophore composition is useful as an immunoassay reagent, while non-target chelate-fluorophore compositions of the present invention are used in screening and selecting antibodies for use in an immunoassay.

The following describes the invention in greater detail:

I. Chelating Agents

Chelating agents useful in preparing fluorescent chelate compositions of the present invention are derivatives of EDTA or DTPA wherein a p-aminobenzyl-, p-isothiocyanatobenzyl or similar reactive side chain substituent is attached at a methylene carbon atom of the polyamine chain. EDTA and DTPA are the second and third members, respectively, of a homologous series of aminopolycarboxylic acids that are well known in the art. The first member of this series, nitrilotriacetic acid (hereafter NTA) has a maximum denticity (number of metal binding sites) of 4, while the fourth member, triethylenetetramine-N,N,N′, N″,N′″,N′″-hexaacetic acid (hereafter TTHA,) has a maximum denticity of 12. When used as targets for antibody production, the hexadentate EDTA and octadentate DTPA structures offer the advantage of forming predominantly 1:1 chelate complexes of high thermodynamic stability with a wide range of metal ions. NTA complexes are often unstable if exposed for protracted periods to typical conditions encountered in vivo, while TTHA and higher members of the series tend to form chelates that have metal:chelator stoichiometries of 2:1 or higher, greatly complicating interpretation of anti-chelate binding data. Of linear, aminopolycarboxylic acid chelating agents, those having the core structure of either EDTA or DTPA are preferred as targets for antibody production.

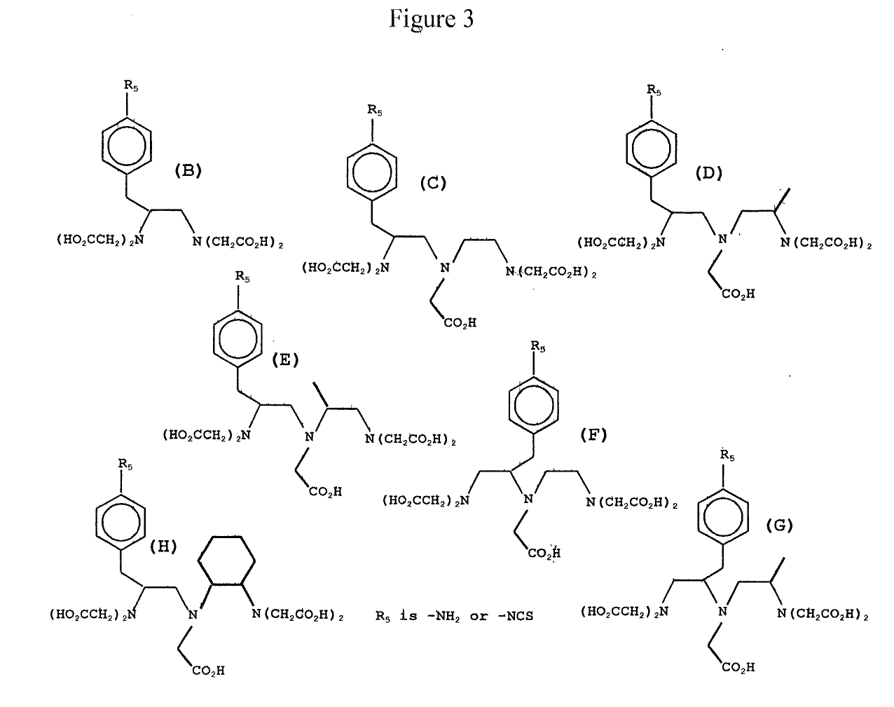

Non-limiting examples of chelating agents useful with the present invention are structures (B)-(H) of FIG. 3. In the case of DTPA derivatives, the reactive substituent may be attached at a carbon atom proximal to the central nitrogen atom (structures (F) and (G) of FIG. 3) or at a carbon atom proximal to a terminal nitrogen atom (structures (B), (C), (D), (E) and (H) of FIG. 3). Methylene carbon atoms of the polyamine chain other than those that bear the p-aminobenzyl-, p-isothiocyanatobenzyl- or other reactive substituent may be optionally substituted with methyl groups (as in structures (D), (E) and (G) of FIG. 3) or may be fused into a ring system (as in structure (H) of FIG. 3). These latter modifications of the core structure of EDTA and DTPA can somewhat increase the kinetic stability of the chelate complexes, but they do not affect the fundamental metal binding properties of such molecules. The essential features of EDTA and DTPA derivatives useful in this invention are (a) retention of the metal binding properties of the parent EDTA and DTPA structures; and (b) the presence of a reactive side chain through which the chelating moiety may be covalently linked to a fluorophore.

II. Fluorophores

Non-limiting examples of fluorescent dye molecules useful in preparing chelate-fluorophore compositions of the present invention are: fluorescein derivatives, Texas Red derivatives, rhodamine derivatives, coumarin derivatives, BODIPY™ dyes (Molecular Probes, Eugene, Oreg.), pyrene derivatives and naphthalene derivatives, with fluorescein derivatives being preferred. Particularly preferred are fluoresceinamine (isomers I and II) and fluorescein isothiocyanate. The essential characteristics a fluorophore must possess to be useful in this invention are (a) a fluorescence lifetime and quantum yield suitable for monitoring hapten-antibody binding at nanomolar concentrations by fluorescence polarization; and (b) the presence of a reactive substituent through which the fluorophore may be covalently linked to an EDTA or DTPA derivative. Preferred fluorophores possess excited state lifetimes of about 2 to about 20 nanoseconds, a molar extinction coefficient of about 20,000 or greater, and a quantum yield preferably greater than about 0.5. Preferred reactive substituents are amine groups, isothiocyanate groups, N-hydroxysuccinimidyl ester groups and dichlorotriazinyl groups.

III. Covalent Linkage of Fluorophore and Chelating Agent

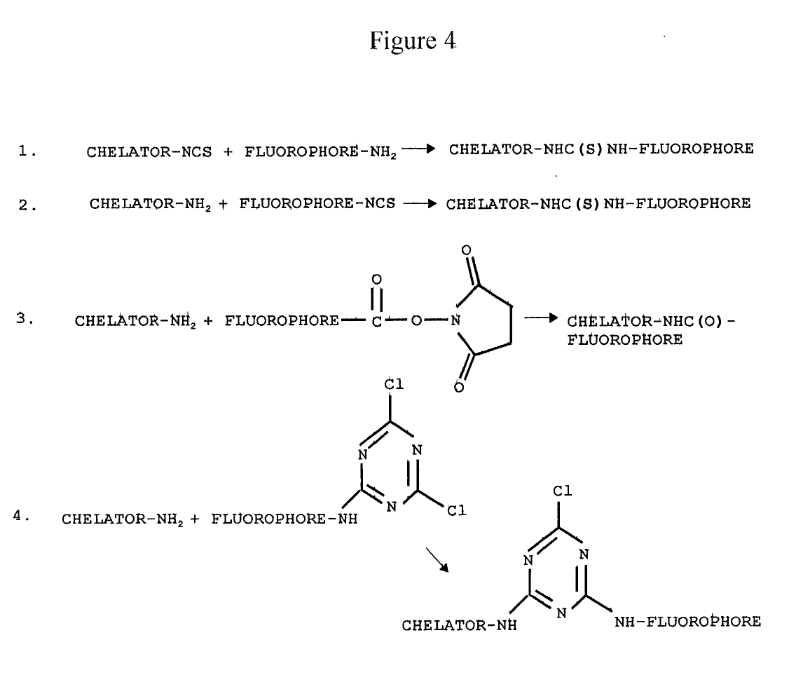

Non-limiting examples of reaction schemes that may be used to covalently link an EDTA or DTPA chelator derivative to a fluorophore appear in FIG. 4. Reaction sequences 1 and 2 of FIG. 4 result in an identical thiourea linkage between the fluorophore and the chelating moiety and are preferred. Alternatively, an amine side chain on the chelating agent may be reacted with an N-hydroxysuccinimidyl ester derivative of a fluorophore to produce an amide linkage (reaction 3 of FIG. 4) or with a dichlorotriazinyl derivative of a fluorophore (reaction 4 of FIG. 4). EDTA and DTPA derivatives bearing p-aminobenzyl and p-isothiocyanatobenzyl side chains are prepared according to Sundberg et al. [J. Med. Chem., 17: 1304-7 (1974)] and Brechbiel et al. [Inorg. Chem., 25: 2772-81 (1986); Bioconjugate Chem., 2:187-94 (1991)]. Useful fluorophores are commercially available as amine derivatives and may also be obtained as isothiocyanates, N-hydroxysuccinimidyl esters and dichlorotriazinyl derivatives by virtue of their widespread use as protein labels. Coupling reactions of the type shown in FIG. 4 are well known, and one skilled in the art will readily select the appropriate derivatives and coupling scheme based on the availability of particular chelating agents and fluorophores and the desired properties of the conjugate.

IV. Complexation with Metal Ion to Give the Chelate-fluorophore Composition

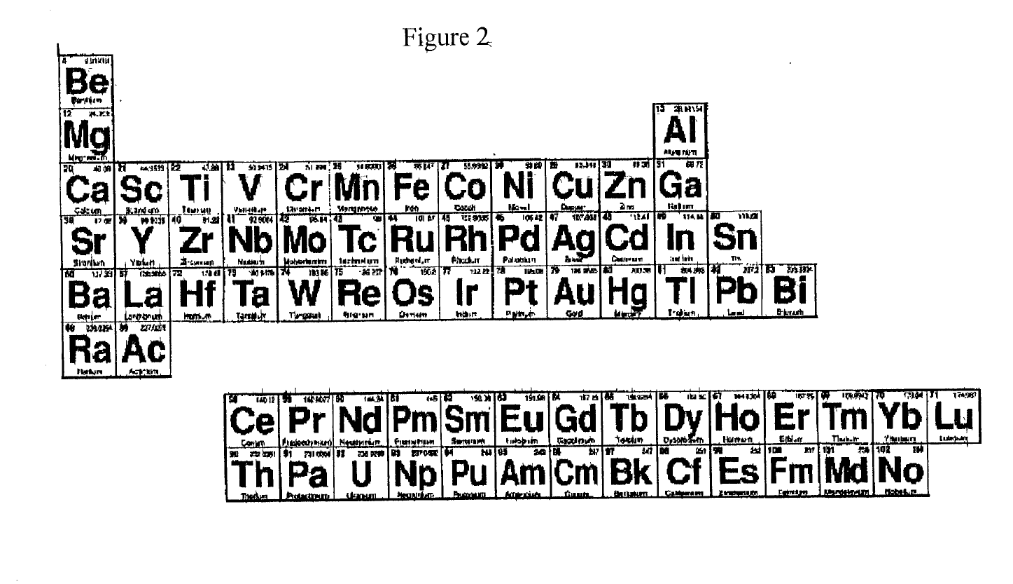

Metal ions of interest to the present invention are shown in FIG. 2, which comprise those elements known to form chelate complexes with EDTA and DTPA. These consist of bismuth, tin, lead, aluminum, gallium, indium, thallium and the members of groups IIa, IIIa, IVa, Va, VIa, VIIa, VIII Ib and VIII IIb of the periodic table of the elements, the lanthanide series of elements and the actinide series of elements other than lawrencium.

Any given metal ion is complexed with an EDTA-fluorophore or DTPA-fluorophore conjugate of the present invention by the steps of:

(a) Dissolving the chelating agent-fluorophore conjugate in water at as high a concentration as possible, preferably greater than about 1 mM; most preferably about 10 mM.

(b) Adding mineral acid until the pH of the solution is at or below about 2.

(c) Dissolving a suitable salt of the metal ion in distilled water to produce a solution of preferably at least about 1 mM and most preferably, about 20 mM.

(d) Adding the metal ion solution to the conjugate solution, with stirring at room temperature at a metal ion:conjugate stoichiometry of about 1.0-1.1:1.0.

(e) Adding aqueous base drop-wise with continued stirring until the pH reaches about 7 or greater.

The resulting solution contains the chelate-fluorophore composition of the given metal ion suitable for subsequent use in screening and selecting anti-chelate antibodies. Until such use, the composition is stored frozen at high concentration and protected from light.

Preferred metal ion salts are nitrates, but chlorides, acetates, sulfates, carbonates, bicarbonates, perchlorates or other salts may be used, depending on availability for any given metal. A preferred mineral acid is hydrochloric acid at a concentration of about 1 M but nitric or sulfuric acids may also be used. A preferred aqueous base for neutralization is about 1 M sodium hydroxide.

It is desirable to carry out the complexation reaction at as high a concentration as is practical, to minimize the chance that adventitious metal contamination of glassware or reagents might result in the chelating moiety in the chelator-fluorophore conjugate becoming occupied by a metal ion other than the one intended. EDTA-fluorophore and DTPA-fluorophore conjugates are generally highly soluble in water at neutral pH, when the carboxylic acid groups are ionized. When the pH is reduced to 2 or below, partial precipitation of the conjugate sometimes occurs as the carboxylate groups become protonated. Such precipitates redissolve as the solution is neutralized, and the final chelate-fluorophore composition is a clear solution. To maximize long term stability during storage, the chelate-fluorophore compositions are stored frozen at high concentration and are diluted into the working concentration range immediately before use.

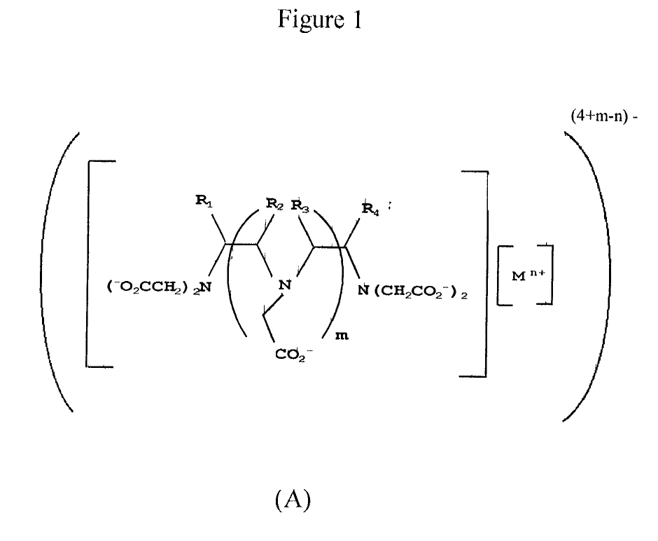

Preferred compositions according to the present invention have structure (A) (FIG. 1) wherein M is a metal selected from those shown in FIG. 2, m is 0, R₄ is H and R₁ is p-CH₂C₆H₄—X—Y wherein X is —HNC(S)NH—, —NHC(O) or —NH—C₃N₃Cl—NH— and Y is a fluorophore.

Other preferred compositions have structure (A) (FIG. 1) wherein M is a metal selected from those shown in FIG. 2, m is 1, R₂ is H, R₃ is H, R₄ is H and R₁ is p-CH₂C₆H₄—X—Y wherein X is —HNC(S)NH—, —NHC(O) or —NH—C₃N₃Cl—NH— and Y is a fluorophore.

Additional preferred compositions have structure (A) (FIG. 1) wherein M is a metal selected from those shown in FIG. 2, m is 1, R₂ is H, R₃ is H, R₄ is CH₃ and R₁ is p-CH₂C₆H₄—X—Y wherein X is —HNC(S)NH—, —NHC(O) or —NH—C₃N₃Cl—NH— and Y is a fluorophore.

Also preferred are compositions according to structure (A) (FIG. 1) wherein M is a metal selected from those shown in FIG. 2, m is 1, R₂ is H, R₃ is CH₃, R₄ is H and R₁ is p-CH₂C₆H₄—X—Y wherein X is —HNC(S)NH—, —NHC(O) or —NH—C₃N₃Cl—NH— and Y is a fluorophore.

Further preferred compositions include those having structure (A) (FIG. 1) wherein M is a metal selected from those shown in FIG. 2, m is 1, R₁ is H, R₃ is H, R₄ is H and R₂ is p-CH₂C₆H₄—X—Y wherein X is —HNC(S)NH—, —NHC(O) or —NH—C₃N₃Cl—NH— and Y is a fluorophore.

Similarly preferred are compositions having structure (A) (FIG. 1) wherein M is a metal selected from those shown in FIG. 2, m is 1, R₁ is H, R₃ is H, R₄ is CH₃ and R₂ is p-CH₂C₆H₄—X—Y wherein X is —HNC(S)NH—, —NNHC(O) or —NH—C₃N₃Cl—NH— and Y is a fluorophore.

Also preferred are compositions having structure (A) (FIG. 1) wherein M is a metal selected from those shown in FIG. 2, m is 1, R₂ is H, R₃ and R₄ are —CH₂CH₂— and are fused into a cyclohexyl ring system and R₁ is p-CH₂C₆H₄—X—Y wherein X is —HNC(S)NH—, —NHC(O) or —NH—C₃N₃Cl—NH— and Y is a fluorophore.

Other preferred compositions according to the present invention have structure (A) (FIG. 1) wherein M is any main group or transition metal shown in FIG. 2, m is 0, R₄ is H and R₁ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Additional preferred compositions have structure (A) (FIG. 1) wherein M is any main group or transition metal shown in FIG. 2, m is 1, R₂ is H, R₃ is H, R₄ is H and R₁ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Also preferred are compositions having structure (A) (FIG. 1) wherein M is any main group or transition metal shown in FIG. 2, m is 1, R₂ is H, R₃ is H, R₄ is CH₃, and R₁ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Additional preferred compositions include those having structure (A) (FIG. 1) wherein M is any main group or transition metal shown in FIG. 2, m is 1, R₂ is H, R₃ is CH₃, R₄ is H, and R₁ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Similarly preferred are compositions having structure (A) (FIG. 1) wherein M is any main group or transition metal shown in FIG. 2, m is 1, R₁ is H, R₃ is H, R₄ is H, and R₂ is p-CH₂C₆H₄—X—Y wherein X is —HNC(S)NH—, —NNHC(O) or —NH—C₃N₃Cl—NH— and Y is fluorescein, Texas Red, rhodamine, coumarin, pyrene, naphthalene or a BODIPY dye.

Other preferred compositions have structure (A) (FIG. 1) wherein M is any main group or transition metal shown in FIG. 2, m is 1, R₁ is H, R₃ is H, R₄ is CH₃ and R₂ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Also preferred are compositions having structure (A) (FIG. 1) wherein M is any main group or transition metal shown in FIG. 2, m is 1, R₂ is H, R₃ and R₄ are —CH₂CH₂— and are fused into a cyclohexyl ring system, and R₁ is p-CH₂C₆H₄—X—Y wherein X is —HNC(S)NH—, —NHC(O) or —NH—C₃N₃Cl—NH— and Y is fluorescein, Texas Red, rhodamine, coumarin, pyrene, naphthalene or a BODIPY dye.

Particularly preferred compositions according to the present invention have structure (A) (FIG. 1) wherein M is any main group or transition metal shown in FIG. 2, m is 0, R₄ is H and R₁ is p-CH₂C₆H₄—X—Y wherein X is —HNC(S)NH— or —NHC(O) or and Y is fluorescein isomer I or fluorescein isomer II.

Other particularly preferred compositions have structure (A) (FIG. 1) wherein M is any main group or transition metal shown in FIG. 2, m is 1, R₂ is H, R₃ is H, R₄ is H, and R₁ is p-CH₂C₆H₄—X—Y wherein X is —HNC(S)NH— or —NHC(O) or and Y is fluorescein isomer I or fluorescein isomer II.

Additional particularly preferred compositions have structure (A) (FIG. 1) wherein M is any main group or transition metal shown in FIG. 2, m is 1, R₂ is H, R₃ is H, R₄ is CH₃, and R₁ is p-CH₂C₆H₄—X—Y wherein X is —HNC(S)NH— or —NHC(O) or and Y is fluorescein isomer I or fluorescein isomer II.

Also particularly preferred are compositions having structure (A) (FIG. 1) wherein M is any main group or transition metal shown in FIG. 2, m is 1, R₂ is H, R₃ is CH₃, R₄ is H, and R₁ is p-CH₂C₆H₄—X—Y wherein X is —HNC(S)NH— or —NHC(O) or and Y is fluorescein isomer I or fluorescein isomer II.

Other particularly preferred compositions have structure (A) (FIG. 1) wherein M is any main group or transition metal shown in FIG. 2, m is 1, R₁ is H, R₃ is H, R₄ is H, and R₂ is p-CH₂C₆H₄—X—Y wherein X is —HNC(S)NH— or —NHC(O) or and Y is fluorescein isomer I or fluorescein isomer II.

Similarly particularly preferred are compositions having structure (A) (FIG. 1) wherein M is any main group or transition metal shown in FIG. 2, m is 1, R₁ is H, R₃ is H, R₄ is CH₃, and R₂ is p-CH₂C₆H₄—X—Y wherein X is —HNC(S)NH— or —NHC(O) or and Y is fluorescein isomer I or fluorescein isomer II.

Another group of particularly preferred compositions have structure (A) (FIG. 1) wherein M is any main group or transition metal shown in FIG. 2, m is 1, R₂ is H, R₃ and R₄ are —CH₂CH₂— and are fused into a cyclohexyl ring system, and R₁ is p-CH₂C₆H₄—X—Y wherein X is —HNC(S)NH— or —NHC(O) or and Y is fluorescein isomer I or fluorescein isomer II.

V. Anti-chelate Antibodies

Antibodies are used as non-limiting examples of biological binding agents that possess a binding site having specificity for a given chelate complex of a given metal ion. Other biological binding agents, such as those produced by phage expression or recombinant antibodies may be similarly screened, selected and used by employing the methods described herein.

Generally, anti-chelate antibodies are produced by methods analogous to those used for any other hapten. The production of antibodies recognizing the complex formed between a particular metal ion and a polyaminopolycarboxylate chelating agent was first disclosed by Meares et al [U.S. Pat. No. 4,722,892 (1988)]and subsequently by Goodwin et al. [J. Nucl. Med., 29: 266-74 (1988)], Le Doussal et al. [Cancer Res., 50: 3445-52 (1989)] and Blake et al. [J. Biol. Chem., 271: 27677-85 (1996)]. To produce an anti-chelate response, test animals are immunized with EDTA or DTPA conjugated to a suitable carrier protein such as keyhole limpet hemocyanin (KLH) or bovine serum albumin (BSA) and complexed with the target metal ion of interest.

VI. Screening Anti-chelate Antibody Responses

To evaluate the immune response, blood samples are periodically drawn and centrifuged to remove the red blood cells. Serial dilutions of the resulting serum in buffer are equilibrated with a fixed concentration, generally between about 1-5 nM with about 2.5 nM preferred, of a target metal chelate-fluorophore composition of the present invention corresponding to the chelate structure with which the animal has been immunized. After brief equilibration, each sample is excited with plane polarized light, and the polarization of the light emitted by the fluorophore is measured using a fluorescence polarization analyzer.

The method relies on using fluorescence polarization as a means of probing rates of molecular rotation in solution at fixed viscosity. The extent to which the emitted light retains the polarization of the light used to excite the fluorophore is proportional to the rate of rotation of the fluorophore during the excited state lifetime and hence to the size of the molecule with which that fluorophore is associated. If the chelate-fluorophore composition is recognized and bound by an antibody in the serum sample, which has a molecular weight of at least about 160 kD, it behaves as a large molecule and the emitted signal retains a significant degree of polarization. If there are no macromolecules in the serum that bind to the chelate-fluorophore composition, which has a molecular weight of less than about 1 kD, it remains free in solution and behaves as a small molecule. In the latter case, rapid tumbling in solution during the excited state lifetime leads to randomization of the spatial orientation of the fluorophore and the emitted light retains little or no polarization.

If an animal fails to mount an immune response to the chelate, polarization values remain low (i.e., unchanged from those obtained when titering serum taken from the same animal before immunization). An animal mounting a response to the chelate produces serum characterized by elevated polarization values.

A serum titer significantly higher than that for a pre-immunization serum sample indicates that a particular animal is producing antibodies that bind to the target chelate. The metal ion specificity of the response can then be evaluated by performing analogous serum titrations against selected non-target chelate-fluorophore compositions of the present invention. These are done using identical serum dilutions and chelate-fluorophore conjugate concentrations as those employed when titering against the target chelate-fluorophore conjugate. In this way, at each data point, a direct comparison is made between the extent of binding to the target chelate vs. the non-target analog, in solution at equilibrium. A difference in observed polarization of 5 mP or greater between target and non-target chelate-fluorophore conjugates indicates a statistically significant difference in antibody-antigen binding at that particular antibody concentration.

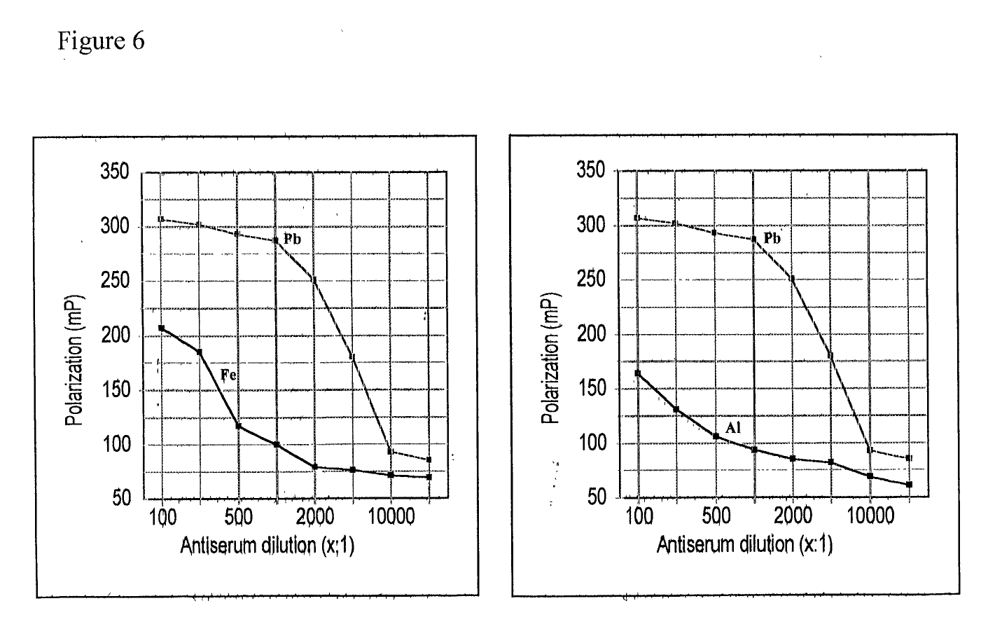

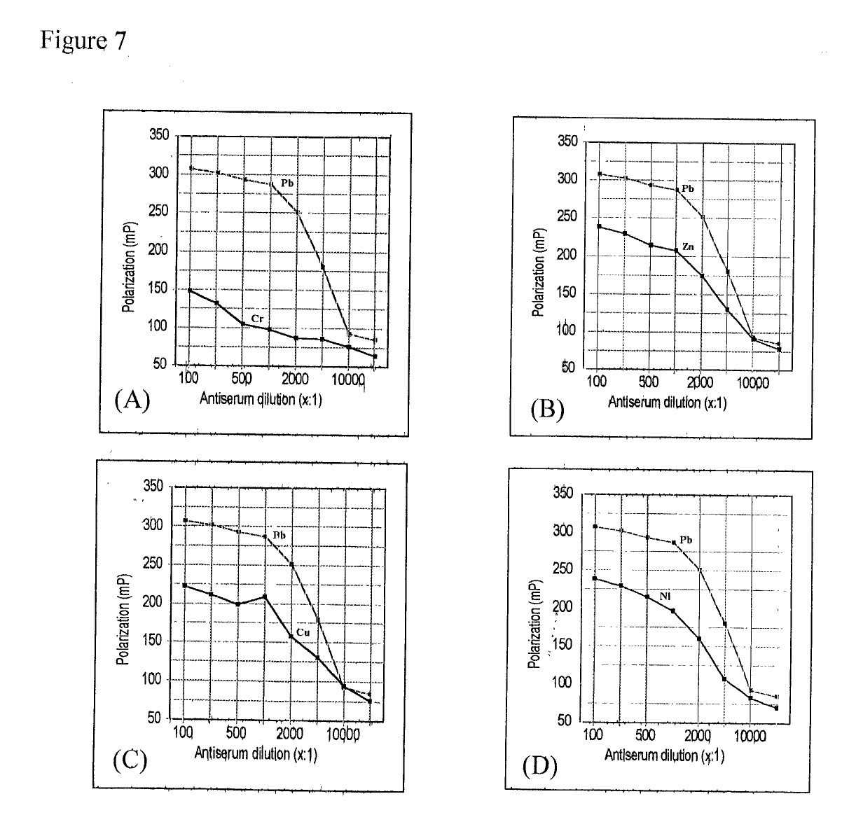

The selection of non-target chelate-fluorophore compositions with which to probe the serum is determined by the application envisioned for the particular anti-chelate antibody. Generally, the ultimate objective is an immunoassay that is not subject to interferences resulting from cross reactivity of the antibody with non-target metals present in the sample. Depending on the type of sample that is being targeted, the nature of non-target metals most likely to be of concern varies. A preferred embodiment of the present invention is to select, for any given application, those non-target metals of greatest concern and to screen antibodies for cross-reactivity with those metals at a very early stage. For example, one aspect of the present invention targets the development of an immunoassay for lead in soil. Of the various chelatable metals typically present in soil two, aluminum and iron, are ubiquitously present at very high concentrations. It is therefore unlikely that an antibody that cross reacted strongly with either aluminum or iron could be used to successfully develop an immunoassay for any other metal in soil samples. Thus, a primary screen of anti-chelate antibodies raised against a lead chelate consists of titering serial dilutions of serum against a fixed concentration of (a) a lead chelate-fluorophore composition of the present invention corresponding to the lead chelate used to immunize the animal, and (b) the corresponding aluminum and iron chelate-fluorophore compositions. Results of this type of screening are illustrated in FIGS. 6 and 7 and are described in greater detail in the EXAMPLES below. A non-specific response is characterized by polarization values for the non-target chelate-fluorophore conjugates that are equal to or exceed those obtained with the analogous target composition. A metal-specific response is characterized by two behaviors: (1) at any given serum dilution, polarization values are higher for the target chelate-fluorophore composition than for the same concentration of non-target analog; and (2) the maximum polarization achieved by the non-target chelate-fluorophore conjugate, even at low serum dilutions, is significantly lower than the maximum polarization seen with the target chelate-fluorophore conjugate.

The nature and number of non-target metals to be screened in this way may be selected based on known sample characteristics and desired immunoassay performance criteria. For example, an anti-chelate antibody targeted toward clinical applications would not need to be screened against aluminum, which is present in physiological fluids in minuscule concentrations, but would still need to be tested for cross-reactivity with iron and probably also against zinc and copper. Similarly, an anti-chelate antibody being developed to monitor silver concentrations in photographic film recycling operations would probably not need to be screened for cross reactivity with main group or first transition series elements but might need to be tested for cross reactivity with other heavy metals. One skilled in the art will readily select non-target metals against which to screen a given antibody based on the known characteristics of the sample matrix in which it is envisioned that antibody will be used.

VII. Immunoassays for Metal Ions Using Anti-chelate Antibodies

Antibodies that bind selectively to a given target metal complex of EDTA or DTPA may be identified and characterized using the screening procedures described herein. Once identified, any given anti-chelate antibody can be used to configure an immunoassay that is useful in measuring concentrations of the target metal in a particular sample matrix. Any immunoassay format that can be configured to measure low molecular weight antigens (haptens)can be modified to measure chelate concentrations.

The first disclosure of an immunoassay that measured a given metal complex of a polyaminopolycarboxylate chelating agent employed rabbit polyclonal antibodies in a radioimmunoassay format [Ogan et al., J. Pharm. Sci., 82: 475-479 (1993)]. Enzyme linked immunosorbent assays that measure concentrations of the In(III)-EDTA chelate and Cd(II)-EDTA complex [Chakrabarti et al. Anal. Biochem., 217: 70-75 (1994); Khosraviani et al. Environ. Sci. Technol., 32: 137-42 (1998)] have subsequently been disclosed. The foregoing assay formats can be readily applied to anti-chelate antibodies selected by means of the present invention, in those applications where a radioisotopic or enzymatic read-out is preferred.

A particularly preferred assay format in which an anti-chelate antibody is used to measure the concentration of a target metal ion is a competitive binding fluorescence polarization immunoassay (FPIA). In this procedure, a fixed concentration of a target chelate-fluorophore composition of the present invention competes for a fixed concentration of antibody binding sites with varying concentrations of target chelate formed by adding excess chelating agent to the sample. The FPIA technique has been widely applied to the measurement of low molecular weight organic analytes and offers advantages of speed and simplicity, as the antibody-antigen reaction is monitored in solution and no washing or color development steps are required. For example, as illustrated and described in greater detail in EXAMPLES 5-8 below, anti-EDTA-Pb antibodies can be used in conjunction with a lead-EDTA-fluorescein conjugate of the present invention to configure FPIA assays that measure lead(II) in the form of its EDTA chelate. Such an FPIA for the pure 1:1 EDTA-Pb chelate in distilled water has a dynamic range of 0-1000 ppt and a limit of detection below 20 ppt.

A preferred embodimemt of the present invention presents FPIA tests for lead in mineral acid extracts from samples of soil and airborne dust. Such mineral acid extractions are commonly used in the art and are the method of choice for preparing samples for conventional atomic absorption or atomic emission spectroscopy analysis. Typical extraction procedures produce final lead concentrations between about 1 ppm and about 100 ppm and final mineral acid concentrations of about 0.1 M to about 2-3 M. Such extracts generally also contain high concentrations of aluminum and iron plus significant amounts of a variety of other non-target metals. In a preferred assay procedure, an aliquot of the mineral acid extract is diluted at least about 100-fold into an assay diluent with consists of an aqueous solution containing a base, a chelating agent and the corresponding fluorophore-target chelate composition. Anti-chelate antibody is then added and after brief incubation at room temperature the polarization is measured. Preferred assay diluents contain between about 10-100 mM sodium bicarbonate or HEPES as base, between about 10-100 μM EDTA or DTPA and between about 1-10 nM fluorophore-target chelate conjugate. Preferred antibodies are those screened for low cross-reactivity with aluminum and iron according to the present invention. A particularly preferred assay diluent contains 25 mM sodium bicarbonate, 25 μM EDTA and 2.5 nM fluorophore-target chelate conjugate (structure (A) of FIG. 1 wherein M is Pb, n is 2, m is 0, R₄ is H and R₁ is p-CH₂C₆H₄—X—Y wherein X is —NHC(S)NH— and Y is fluorescein). A particularly preferred antibody is a rabbit polyclonal antiserum raised against the EDTA-Pb complex and screened for cross reactivity with aluminum, iron, chromium, zinc, copper and nickel.

The foregoing preferred assays measure lead in mineral acid extracts of soils and airborne dust by intentionally diluting the sample during the course of mixing same with the assay reagents. Dilutions of from about 100:1 to about 100,000:1 may be usefully employed in this assay format. Another aspect of the present invention presents FPIA methods for measuring lead in an aqueous sample wherein sample dilution is minimized. Such procedures are preferred when lead concentrations in a given sample matrix are expected to be low, e.g., drinking water, where lead levels should be below 15 ppb. In such situations, a preferred assay procedure described in detail in EXAMPLE 7, entails adding no more than about 0.2 mL of reagents to about 2.0 mL of sample so that the maximum dilution of lead in the sample as a result of adding all necessary immunoassay reagents is about 10% at most.

FPIA assays performed according to the present invention using anti-chelate antibodies selected as disclosed herein do not appear to be subject to major interferences arising from cross-reactivity with non-target metals.

Also provided by the present invention are immunoassay test kits which contain all of the FPIA reagents needed to perform the assay together with calibrators, controls or other vendor-supplied materials as required.

The foregoing considerations may be further illustrated by the following examples, which are intended for purposes of illustration only and should not be construed in any sense as limiting the scope of the invention.

### EXAMPLE 1

### Preparation of a Chelator-fluorophore Conjugate

In this example, a chelator-fluorophore conjugate comprising EDTA linked to fluorescein is obtained by reaction of a benzylisothiocyanate derivative of the chelating agent with fluoresceinamine (i.e., reaction path 1 of FIG. 4).

Benzylisothiocyanate-EDTA (structure (B) of FIG. 3 wherein R₅ is —SCN) (100 mg, 0.23 mmol) was dissolved in 1 M bicarbonate buffer, pH 8.0 (2.0 ml). The resulting solution was stirred at room temperature, and a solution of fluoresceineamine isomer I (80 mg, 0.23 mmol) in anhydrous DMSO (1.0 mL) was immediately added drop-wise, producing an color change from pale yellow to deep orange-red. The reaction solution was stirred at room temperature for a further 24 hours, then diluted with distilled water to a final volume of 23 mL. The resulting 10 mM stock solution of the chelator-fluorophore conjugate was stored at −20° C. protected from light until needed.

### EXAMPLE 2

### Preparation of Chelate-fluorophore Conjugate Compositions

In this example, aliquots of the chelator-fluorophore composition prepared in Example I are complexed with various metal ions to give the corresponding chelate-fluorophore compositions. Aqueous stock solutions of the metal ions are prepared immediately before use by dissolving weighed amounts of a solid metal salt in distilled water.

### Example 2a

A 20 mM aqueous stock solution of lead(II) was obtained by dissolving lead(II) nitrate (3.31 g) in distilled water (500 ml). A 1.0 mL aliquot of the 10 mM chelator-fluorophore stock prepared in Example 1 was stirred as concentrated HCl (0.2 mL) was added drop-wise, giving a straw colored solution. Stirring was continued as 0.5 mL of the 20 mM lead(II) stock was added, followed by drop-wise addition of 1 M aqueous NaOH to a final pH of about 8, to give a clear yellow solution. The reaction solution was diluted to 100 mL with distilled water to give a 0.1 mM stock solution of chelate-fluorophore conjugate (Structure (A) of FIG. 1 wherein M is Pb, m is 0, n is 2, R₄ is H and R₁ is p-CH₂C₆H₄—X—Y wherein X is —NHC(S)NH— and Y is fluorescein isomer I).

### Example 2b

Ferric chloride hexahydrate (5.40 g) was dissolved in distilled water (1 L) to give a 20 mM stock. A 1.0 mL aliquot of the 10 mM chelator-fluorophore stock prepared in Example 1 was stirred and concentrated HCl (0.2 mL) was added drop-wise. Stirring was continued as 0.5 mL of the 20 mM iron(III) stock was added, followed by drop-wise addition of 1 M aqueous NaOH to a final pH of about 8. The resulting clear yellow reaction solution was diluted to 100 mL with distilled water to give a 0.1 mM stock solution of the chelate-fluorophore conjugate (structure (A) of FIG. 1 wherein M is Fe, n is 3, m is 0, R₄ is H and R₁ is p-CH₂C₆H₄—X—Y wherein X is —NHC(S)NH— and Y is fluorescein isomer I).

### Example 2c

A 20 nM stock solution of Al(III) was prepared by dissolving aluminum trichloride monohydrate (1.55 g) in distilled water (500 mL). A 1.0 mL aliquot of the 10 mM chelator-fluorophore stock prepared in Example 1 was stirred and concentrated HCl (0.2 mL) was added drop-wise. Stirring was continued as a 0.5 mL aliquot of the 20 mM aluminum(III) stock was added, followed by drop-wise addition of 1 M aqueous NaOH to a final pH of about 8. Addition of the aluminum stock produced some precipitate, but this redissolved on addition of the base to give a clear yellow reaction solution. This was diluted to 100 mL with distilled water to give a 0.1 mM stock solution of the chelate-fluorophore conjugate (structure (A) of FIG. 1 wherein M is Al, n is 3, m is 0, R₄ is H and R₁ is p-CH₂C₆H₄—X—Y wherein X is —NHC(S)NH— and Y is fluorescein isomer I).

### Example 2d

Chromium trichloride hexahydrate (2.66 g) was dissolved in distilled water (500 mL) to give a 20 mM stock. A 1.0 mL aliquot of the 10 mM chelator-fluorophore stock prepared in Example 1 was stirred and concentrated HCl (0.2 mL) was added drop-wise. Stirring was continued as a 0.5 mL aliquot of the 20 mM chromium(III) stock was added, followed by drop-wise addition of 1 M aqueous NaOH to a final pH of about 8. The resulting clear yellow reaction solution was diluted to 100 mL with distilled water to give a 0.1 mM stock solution of the chelate-fluorophore conjugate (structure (A) of FIG. 1 wherein M is Cr, n is 3, m is 0, R₄ is H and R₁ is p-CH₂C₆H₄—X—Y wherein X is —NHC(S)NH— and Y is fluorescein isomer I).

### Example 2e

Copper sulfate pentahydrate (2.50 g) was dissolved in distilled water (500 ml) to give a 20 mM stock solution. A 1.0 mL aliquot of the 10 mM chelator-fluorophore stock prepared in Example 1 was stirred as concentrated HCl (0.2 mL) was added drop-wise. Stirring was continued as a 0.5 mL aliquot of the 20 nM copper(II) stock was added, followed by addition of 1 M aqueous NaOH to a final pH of about 8. The resulting clear yellow reaction solution was diluted to 100 mL with distilled water to give a 0.1 mM stock solution of the chelate-fluorophore conjugate (structure (A) of FIG. 1 wherein M is Cu, n is 2, m is 0, R₄ is H and R₁ is p-CH₂C₆H₄—X—Y wherein X is —NHC(S)NH— and Y is fluorescein isomer I).

### Example 2f

Zinc dinitrate hexahydrate (2.97 g) was dissolved in distilled water (500 mL) to give a 20 mM stock solution. A 1.0 mL aliquot of the 10 mM chelator-fluorophore stock prepared in Example 1 was stirred as concentrated HCl (0.2 mL) was added drop-wise. Stirring was continued as a 0.5 mL aliquot of the 20 mM zinc(II) stock was added, followed by addition of 1 M aqueous NaOH to a final pH of about 8. The resulting clear yellow reaction solution was diluted to 100 mL with distilled water to give a 0.1 mM stock solution of the chelate-fluorophore conjugate (structure (A) of FIG. 1 wherein M is Zn, n is 2, m is 0, R₄ is H and R₁ is p-CH₂C₆H₄—X—Y wherein X is —NHC(S)NH— and Y is fluorescein isomer I).

### Example 2g

Nickel(II) chloride hexahydrate (1.19 g) was dissolved in distilled water 250 mL) to give a 20 mM stock solution. A 1.0 mL aliquot of the 10 mM chelator-fluorophore stock prepared in Example 1 was stirred as concentrated HCl (0.2 mL) was added drop-wise. Stirring was continued as a 0.5 mL aliquot of the 20 mM nickel(II) stock was added, followed by addition of 1 M aqueous NaOH to a final pH of about 8. The resulting clear yellow reaction solution was diluted to 100 mL with distilled water to give a 0.1 mM stock solution of the chelate-fluorophore conjugate (structure (A) of FIG. 1 wherein M is Ni, n is 2, m is 0, R₄ is H and R₁ is p-CH₂C₆H₄—X—Y wherein X is —NHC(S)NH— and Y is fluorescein isomer I).

All 0.1 mM stock solutions of target and non-target chelate-fluorophore compositions were stored at −20° C. protected from light until needed.

### EXAMPLE 3

### Immunization of Rabbits with the Lead(II) Chelate of EDTA

Polyclonal antisera against the lead(II)-EDTA complex were produced by standard methods employing immunogens analogous to those previously used to make antibodies against indium(III)-EDTA [Meares et al., U.S. Pat. No. 4,722,892], cobalt(II)-EDTA [Goodwin et al., J. Nucl. Med., 29:226-34 (1988)] and cadmium(II)-EDTA complexes [Blake et al., J.Biol.Chem., 271:27677-85 (1996)].

An immunogen was produced by reacting benzylisothiocyanate-EDTA (50 mg, structure (B) of FIG. 3 wherein R₅ is —SCN) with bovine serum albumin (60 mg) in 0.1M KH₂PO₄/0.1M NaHCO₃, pH 8.5, (5 mL) for 24 hours at 25° C., followed by dialysis into 0.1 M iminodiacetic acid/0.05 M citric acid, pH 6 (hereafter termed “loading buffer”). Excess Pb(NO₃)₂, dissolved in loading buffer, was added, and the resulting solution was incubated for 24 hours at 25° C., then redialyzed extensively against loading buffer. The resulting immunogen, at a final concentration of 1 mg/mL, was emulsified with Freund's adjuvant (complete adjuvant for the primary immunization, incomplete adjuvant for booster doses) and administered to female New Zealand white rabbits (4-6 months old) via multiple subcutaneous injections along each flank. The primary dose contained 1 mg of immunogen, while booster doses contained 0.1 mg of the same material. The animals were bled periodically via a peripheral ear vein, and the serum tested for the presence of antibodies as described below.

### EXAMPLE 4

### Screening Antibodies for Metal Selectivity

In this example, serum from rabbits immunized with the lead(II) chelate of EDTA according to Example 3 are tested for the presence of anti-chelate antibodies by titering serial dilutions of serum in buffer against a fixed concentration of the target chelate-fluorophore composition that was prepared in Example 2a. Sera that show binding to the target chelate, by displaying elevated polarization values out to high serum dilutions, are then titered against the same fixed concentration of the non-target chelate-fluorophore compositions prepared in Examples 2b-2g, and the results obtained with target and non-target compositions are compared. In this example, the antibody is being developed for use in testing soil samples for the presence of lead(II) so a primary screen for non-target reactivity employs aluminum(III) and iron(III) chelate-fluorophore compositions, as these two metals are present at high concentrations in most soils and therefore pose the biggest cross-reactivity challenge. A secondary screen employs four additional non-target metals (chromium(III), copper(II), zinc(II) and nickel(II)) that can be present in soil at lower, but still substantial, concentrations and have chemical properties that are in some respects similar to those of lead(II).

Aliquots of the 0.1 mM target and non-target chelate-fluorophore stock solutions that were prepared in Examples 2a-2g were thawed and diluted with 0.01 M sodium phosphate buffered normal saline, pH 7.4, containing 0.1% sodium azide (hereafter PBSA) to a final concentration of 0.5 μM.

Titrations were performed in 5 mL disposable borosilicate glass test tubes. To each tube was added sequentially (i) 2.0 mL PBSA, (ii) an aliquot of either neat rabbit serum or serum diluted into PBSA: 20 μL neat serum (final dilution 100:1); 10 μL neat serum (final dilution 200:1); 20 μL of a 1:5 dilution of serum in PBSA (final dilution 500:1); 10 μL of a 1:5 dilution of serum in PBSA (final dilution 1,000:1); 50 μL of a 1:50 dilution of serum in PBSA (final dilution 2,000:1); 25 μL of a 1:50 dilution of serum in PBSA (final dilution 4,000:1); 10 μL of a 1:50 dilution of serum in PBSA (final dilution 10,000:1) or 5 μL of a 1:50 dilution of serum in PBSA (final dilution 20,000:1) and (iii) 10 μL of a 0.5 μM chelate-fluorophore stock. The tubes were incubated at room temperature for 10 minutes, then transferred to a fluorescence polarization analyzer (FPM-1, Jolley Consulting & Research, Inc., Grayslake, Ill.) and duplicate readings of the polarization of each tube were recorded. The mean polarization was then calculated and plotted against serum dilution to provide a titer curve.

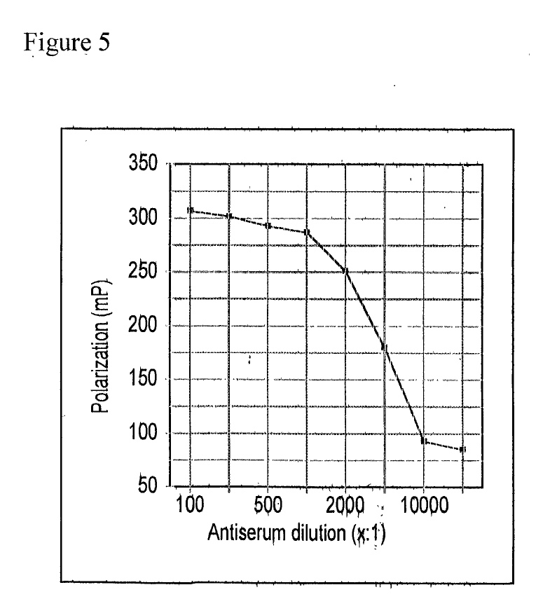

The initial titration study employed the target chelate-fluorophore stock prepared in Example 2a and was used to identify antisera that bound strongly to the target EDTA-Pb(II)chelate. Such a response is illustrated in FIG. 5. Those antisera that displayed such behavior then underwent primary screening for non-target metal cross-reactivity by titration against the iron(III) and aluminum(III) chelate-fluorophore compositions prepared in Examples 2b and 2c. Antisera that showed preferential binding to the EDTA-lead(II)chelate, as illustrated in FIG. 6, were further evaluated by titration against the chromium(III), copper(II), zinc(II) and nickel(II) chelate fluorophore compositions prepared in Examples 2d-2g. Antisera that also displayed preferential binding to the EDTA-Pb(II) chelate relative to non-target chelates in the secondary screen, as illustrated in FIG. 7, became candidates for subsequent use in developing an immunoassay for lead(II).

The precision with which any one point on the titer curves used in this example could be measured was studied, so as to define the minimum difference in polarization produced by target and non-target compositions that could be considered statistically significant. For this purpose, five replicate tubes were prepared for each composition at a 2,000:1 dilution of the antiserum used to produce the Pb vs. Zn data shown in FIG. 7(B), and five replicate polarization readings were made on each tube. For the lead(II) chelate-fluorophore composition, the mean polarization was 264 mP and the standard deviation was ±1.3 mP, while for the zinc(II) composition the mean polarization was 176 mP and the standard deviation was ±2.6 mP. The use of 5 tubes/5 reads, as opposed to the one tube/2 reads protocol normally employed, produced little difference in the average polarization value obtained (264 vs. 251 mP for Pb and 176 vs. 174 mP for Zn). For polarization differences of 10 mP and greater, the one tube/2 read protocol is adequate to establish a significant difference in target chelate vs. non target chelate binding. Differences of 5-10 mP in the polarization produced by target vs. non-target compositions, while small, nevertheless probably represent a real difference in binding but it may require additional replicate tubes and replicate reads to confirm this.

### EXAMPLE 5

### Immunoassay for a Metal Ion Using a Metal-selective Anti-chelate Antibody: Lead in Soil

In this example, an antibody that selectively binds to the EDTA-Pb(II) complex while showing low reactivity with the analogous iron(III) and aluminum(III) chelates is used to configure an FPIA for lead in soil. Soil samples are first treated with a strong mineral acid, a procedure long used in the art for extracting metals into a solution phase in ionic form suitable for analytical measurements. The soil extracts are analyzed by conventional flame atomic absorption spectroscopy (hereafter AAS) and by FPIA, using the target chelate-fluorophore composition prepared in Example 2a and a rabbit antiserum selected as described in Example 4. Linear regression analysis is performed to evaluate the extent to which the FPIA results correlate with the AA values.

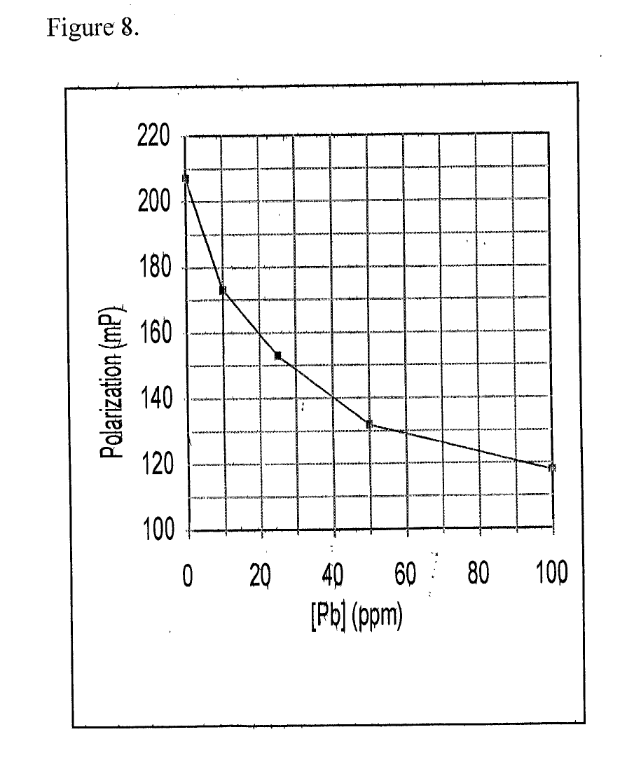

Soil samples were dried in an oven to constant weight and were sieved through a 2 mm screen. Each sample (5 g) was stirred with 1 M nitric acid (50 mL) for one hour at room temperature, then filtered. One aliquot of the filtrate was taken for AAS measurement of its lead content. A second aliquot of the filtrate (100 μL) was diluted with distilled water (2.0 mL), and an aliquot of the resulting dilute extract (20 μL) was added to an aliquot of assay diluent (2.0 mL) in a 5 mL disposable borosilicate glass test tube. The assay diluent was prepared by combining distilled water (200 mL), 1 mM aqueous EDTA (5 mL), 1 M aqueous sodium bicarbonate (5 mL) and a 0.5 μM stock solution of the lead chelate-fluorophore composition prepared in Example 2a (1.0 mL), then adjusting the pH of the resulting solution to about 7.0 by addition of 1 M nitric acid. An aliquot of dilute antiserum (30 μL at a dilution in PBSA that had been determined by prior titration to produce about 80% of maximum polarization) was added and the tube contents were mixed briefly then allowed to stand at room temperature for 30 minutes. Each tube was then transferred to a fluorescence polarization analyzer (FPM-1), and duplicate polarization readings were made. The mean polarization for each tube was calculated, and the lead content of the nitric acid extract was determined from a standard curve that related polarization to lead concentration for pure ionic lead(II) standards (Aldrich Chemical Co., Milwaukee, Wis.) in 1 M nitric acid. The standard curve is shown in FIG. 8. The lead content of each soil sample was then calculated by multiplying the concentration in the nitric acid extract by a factor of 10.

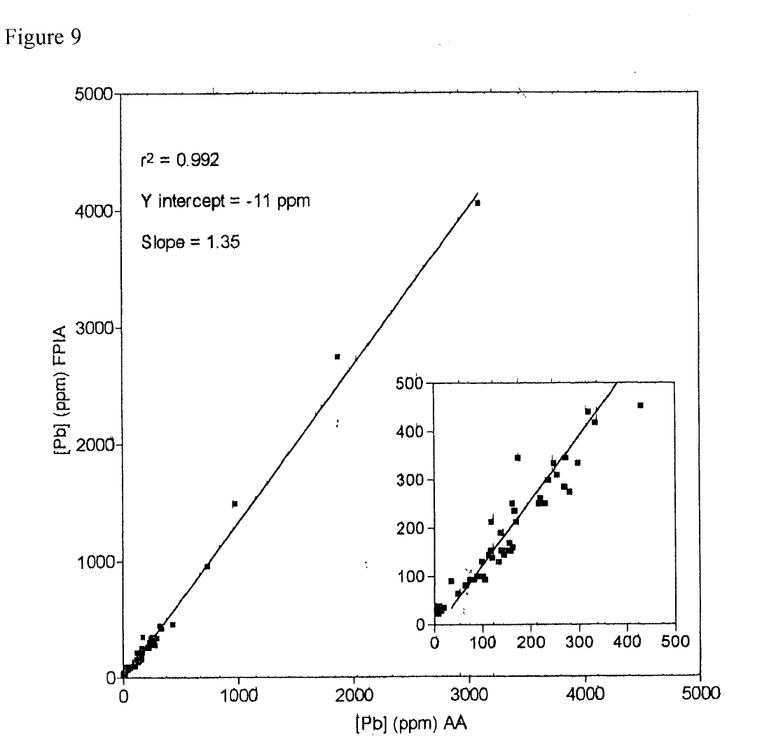

A total of 55 soil samples with lead content ranging from about 40 ppm to about 3,000 ppm were analyzed in this way by both AA and FPIA. These data appear in FIG. 9.

### EXAMPLE 6

### Immunoassay for Lead in Acid Extracts from Airborne Particulates

Samples used in the study consisted of airborne particulates collected on a fiberglass filter by a standard protocol in the art for collection and quantitation of particulate matter with a mean diameter of 10 microns or greater (PM10). Lead had been solubilized by a standard protocol, which entailed extracting the filter with a mixture obtained by combining 26 mL conc. HNO₃ with 11.6 mL conc. HCl and diluting with distilled water to a final volume of 1 L, hereafter termed “acid matrix” (total acid content of approximately 0.4M). One aliquot of the extract was taken for lead measurement by AAS. Splits of each extract were received for immunoassay analysis, including nine samples that were negative (samples 1-9) and six negative samples that had been spiked to a final lead concentration of 3 mg/L (samples 10-15). There were no positive samples available from this collection; the only positive samples tested were the six artificially positive spiked extracts.

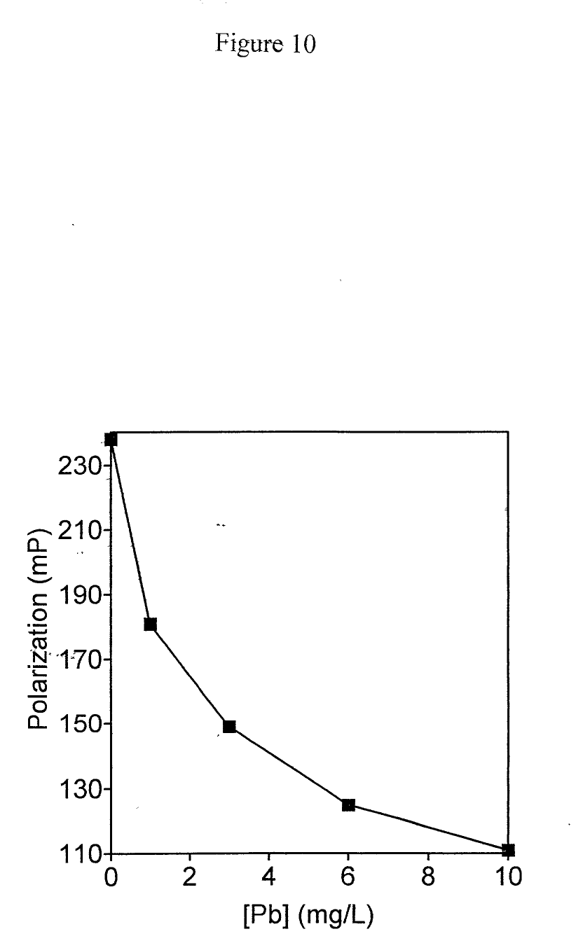

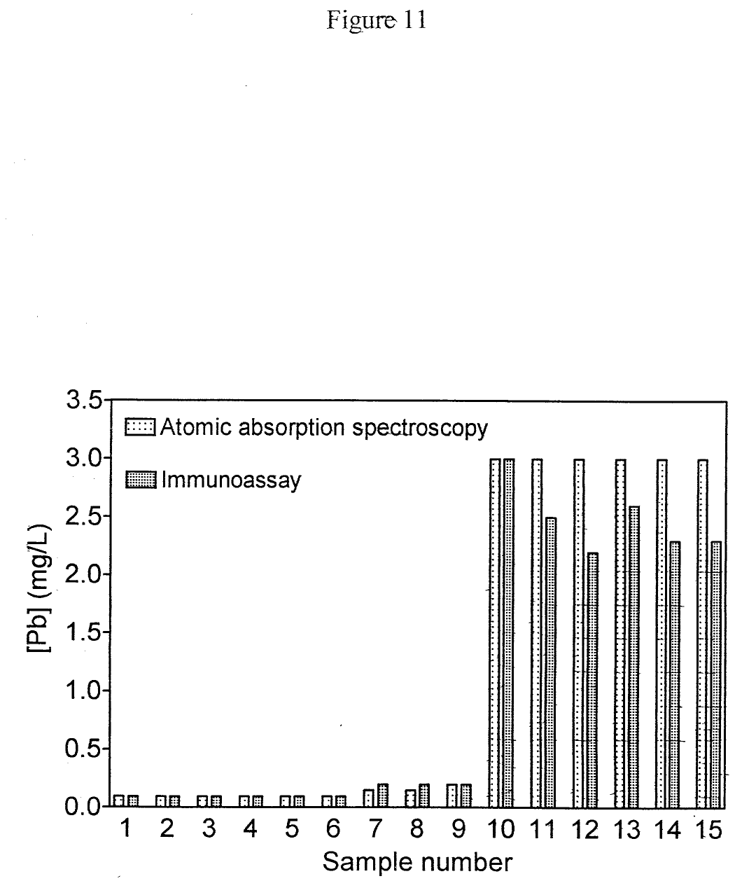

1 mM EDTA (2.5 mL) and 1 M NaHCO₃ (2.5 mL) were combined and diluted with distilled water to a final volume of 100 mL, then a 0.5 μM stock solution of the lead chelate-fluorophore conjugate prepared according to Example 2a (0.5 mL) was added. The resulting solution is hereafter termed “diluent stock”. The immunoassay was performed in 5 mL disposable borosilicate glass test tubes by pipetting into each tube, sequentially: (a) 2.0 ml diluent stock; (b) 20 μL of acid extract or standard; and (c) 20 μL of a 1:10 dilution of antiserum in PBSA (an amount previously determined to give about 80% of maximum polarization by titration according to Example 4). The tubes were incubated at 25° C. for 30 min. then read in duplicate using an FPM-1 analyzer The mean polarization was calculated, and the lead concentration in the extract was determined from a standard curve relating observed polarization to lead content. Lead standards were prepared by diluting a lead AAS standard into acid matrix. The standard curve is shown in FIG. 10. Results obtained by AAS and FPIA for the nine negative and six spiked negative extracts appear in FIG. 11.

### EXAMPLE 7

### Immunoassay of Lead in Drinking Water

In this example municipal drinking water is spiked with a Pb AAS standard to various final lead concentrations. The spiked samples are then analyzed by FPIA under conditions that minimize dilution of the sample.

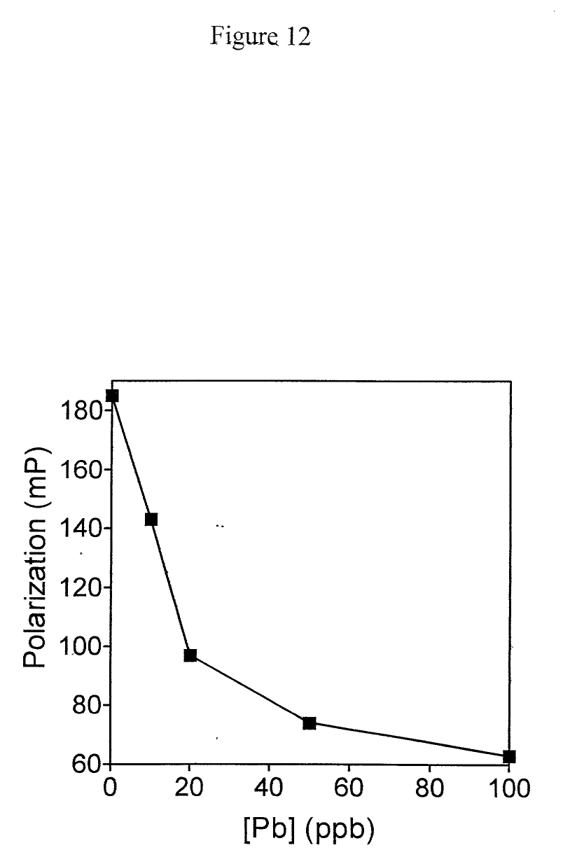

Municipal tap water (Grayslake, Ill.) was spiked with a 1000 ppm Pb AAS standard (Aldrich) to final lead concentrations of 10, 20, 50 and 100 ppb. (The ambient lead concentration of the tap water was less than 1 ppb.) Aliquots (2.0 mL) of spiked tap water were pipetted into 5 mL disposable glass test tubes. To each tube was added, sequentially, (a) 1 mM EDTA (50 μL); (b) a 0.5 μM stock solution of the lead chelate-fluorophore conjugate prepared according to Example 2a (10 μL); and (c) rabbit antiserum obtained according to Examples 3 and 4 in PBSA at a dilution previously determined by titration, according to Example 4, to produce about 80% of maximum polarization (60 μL). Each tube was incubated at room temperature for 15 minutes, then duplicate polarization measurements were made using an FPM-1 analyzer. The mean polarization was calculated and plotted against the concentration of added lead(II). The results are shown in FIG. 12.

### EXAMPLE 8

### Immunoassay of Pure EDTA-Pb Chelate

In this example, the pure 1:1 complex of lead(II) with EDTA is analyzed in the absence of either free EDTA or non-target metals, to assess the absolute sensitivity with which the assay is able to detect the target chelate.

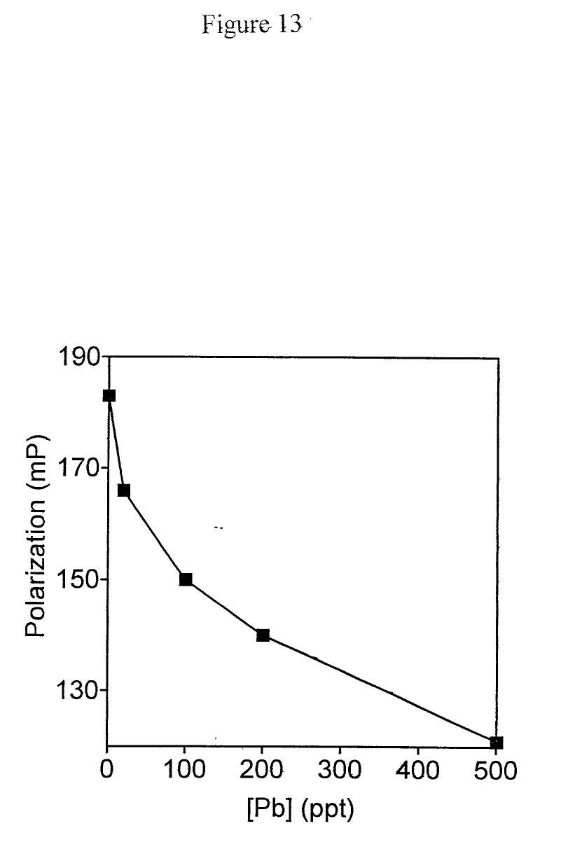

A set of EDTA-Pb standards was prepared by dilution of a 1 mM stock solution with distilled water to give solutions with final lead concentrations of 0, 20, 100, 200 and 500 ppt. Aliquots (2.0 mL) of each standard were transferred to 5 mL disposable borosilicate glass test tubes. To each tube was added, sequentially, (a) a 0.5 μM stock solution of the lead chelate-fluorophore conjugate prepared according to Example 2a (10 μL); and (b) rabbit antiserum, obtained according to Examples 3 and 4, at a dilution in PBSA determined by prior titration, according to Example 4, to produce about 80% of maximum polarization (50 μL). The tubes were incubated for 15 minutes at room temperature, then duplicate polarization measurements were made using an FPM-1 analyzer. The mean polarization was calculated and plotted against the chelate concentration in the standard. These results appear in FIG. 13.

All publications and patent applications mentioned in this patent application are herein incorporated by reference to the same extent as if each of them had been individually indicated to be incorporated by reference.

Although the foregoing invention has been described in some detail by way of illustration and example for purposes of clarity and understanding, it will be obvious that various modifications and changes which are within the skill of those skilled in the art are considered to fall within the scope of the appended claims. Future technological advancements which allow for obvious changes in the basic invention herein are also within the claims. Various modifications of the invention in addition to those shown and described herein which are apparent to those skilled in the art from the preceding description are considered to fall within the scope of the appended claims.

I claim:

1. A chelate-fluorophore tracer composition comprising:
a metal-chelated reagent having the formula 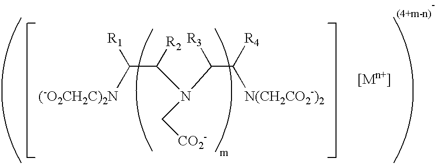
wherein m is 0 or 1; n is 1, 2, or 3; R₁ is p-CH₂C₆H₄—X—Y or H, R₂ is H or p-CH₂C₆H₄—X—Y, and R₃ and R₄ are H, CH₃, or are fused into a ring system; X is —HNC(S)NH—, —NHC(O)— or —NH—C₃N₃Cl, —NH—; Y is a fluorophore having a fluorescence lifetime and quantum yield suitable for monitoring hapten-antibody binding at nanomolar concentrations by fluorescence polarization; and M is a metal chelated thereto selected from the group consisting of bismuth, tin, lead, aluminum, gallium, indium, thallium, elements of Groups IIa, IIIa, IVa, Va, VIa, VIIa, VIII Ia, and VIII Ib of the Periodic Table of the Elements, elements of the lanthanide series of the Periodic Table of the Elements, and elements of the actinide series of the Periodic Table of the Elements, excluding lawrencium.

2. A chelate-fluorophore tracer composition comprising:
a metal-chelated reagent having the formula 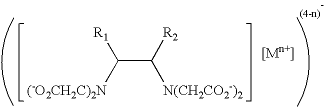
wherein n is 1, 2, or 3; R₁ is p-CH₂C₆H₄—X—Y, R₂ is H; X is —HNC(S)NH—, —NHC(O)— or —NH—C₃N₃Cl—NH—; Y is a fluorophore having a fluorescence lifetime and quantum yield suitable for monitoring hapten-antibody binding at nanomolar concentrations by fluorescence polarization; and M is a metal chelated thereto selected from the group consisting of bismuth, tin, lead, aluminum, gallium, indium, thallium, elements of Groups IIa, IIIa, IVa, Va, VIa, VIIa, VIII Ia, and VIII Ib of the Periodic Table of the Elements, elements of the lanthanide series of the Periodic Table of the Elements, and elements of the actinide series of the Periodic Table of the Elements, excluding lawrencium.

3. A chelate-fluorophore tracer composition comprising:
a metal-chelated reagent having the formula 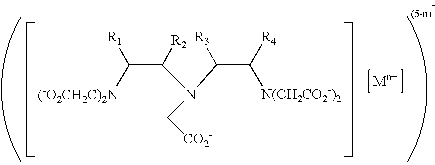
wherein n is 1, 2, or 3; R₁ is p-CH₂C₆H₄—X—Y or H, R₂ is H or p-CH₂C₆H₄—X—Y, and R₃ and R₄ are H, CH₃, or are fused into a ring system; X is —HNC(S)NH—, —NHC(O)— or —NH—C₃N₃Cl—NH—; Y is a fluorophore having a fluorescence lifetime and quantum yield suitable for monitoring hapten-antibody binding at nanomolar concentrations by fluorescence polarization; and M is a metal chelated thereto selected from the group consisting of bismuth, tin, lead, aluminum, gallium, indium, thallium, elements of Groups IIa, IIIa, IVa, Va, VIa, VIIa, VIII Ia, and VIII Ib of the Periodic Table of the Elements, elements of the lanthanide series of the Periodic Table of the Elements, and elements of the actinide series of the Periodic Table of the Elements, excluding lawrencium.

4. The chelate-fluorophore tracer composition of claim 2 or 3, further comprising the metal-chelated reagent wherein Y is selected from the group consisting of fluorescein derivatives, Texas red derivatives, rhodamine derivatives, coumarin derivatives, pyrene derivatives, naphthalene derivatives, and BODIPY dyes.

5. The chelate-fluorophore tracer composition of claim 2 or 3, further comprising the metal-chelated reagent wherein M is a metal chelated thereto selected from the group consisting of bismuth, tin, lead, aluminum, gallium, indium, thallium, and elements of Groups IIa, IIIa, IVa, Va, VIa, VIIa, VIII Ia, and VIII Ib of the Periodic Table of the Elements.

6. The chelate-fluorophore tracer composition of claim 2 or 3, further comprising the metal-chelated reagent wherein M is a metal chelated thereto selected from the group consisting of bismuth, tin, lead, aluminum, gallium, indium, thallium, cadmium, mercury, chromium, silver, antimony, barium, beryllium, thorium, zirconium, vanadium, nickel, molybdenum, manganese, zinc, cobalt, iron, and copper.

7. A method for preparing a chelate-fluorophore tracer composition comprising:
a) adding a solution of a metal ion selected from the group consisting of bismuth, tin, lead, aluminum, gallium, indium, thallium, elements of Groups IIa, IIIa, IVa, Va, VIa, VIIa, VIII Ia, and VIII Ib of the Periodic Table of the Elements, elements of the lanthanide series of the Periodic Table of the Elements, and elements of the actinide series of the Periodic Table of the Elements, excluding lawrencium, to an acidic solution of a fluorophore tracer composition comprising a chelating reagent having the formula 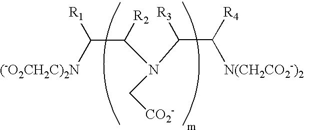
wherein m is 0 or 1, R₁ is p-CH₂C₆H₄—X—Y or H, R₂ is H or p-CH₂C₆H₄—X—Y, and R₃ and R₄ are H, CH₃, or are fused into a ring system; X is —HNC(S)NH—, —NHC(O)— or —NH—C₃N₃Cl—NH—; and Y is a fluorophore having a fluorescence lifetime and quantum yield suitable for monitoring hapten-antibody binding at nanomolar concentrations by fluorescence polarization; and
b) adjusting the pH of the resulting solution to about 7 or greater.

8. A method for preparing a chelate-fluorophore tracer composition comprising:
a) adding a solution of a metal ion selected from the group consisting of bismuth, tin, lead, aluminum, gallium, indium, thallium, elements of Groups IIa, IIIa, IVa, Va, VIa, VIIa, VIII Ia, and VIII Ib of the Periodic Table of the Elements, elements of the lanthanide series of the Periodic Table of the Elements, and elements of the actinide series of the Periodic Table of the Elements, excluding lawrencium, wherein the concentration of said metal ion in the solution is in the range from about 1 mM to about 20 mM, to a second, acidic solution of a fluorophore tracer composition comprising a chelating reagent having the formula 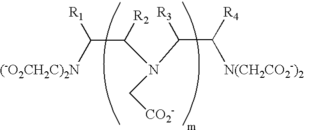
wherein m is 0 or 1, R₁ is p-CH₂C₆H₄—X—Y or H, R₂ is H or p-CH₂C₆H₄—X—Y, and R₃ and R₄ are H, CH₃, or are fused into a ring system; X is —HNC(S)NH—, —NHC(O)— or —NH—C₃N₃Cl—NH—; and Y is a fluorophore having a fluorescence lifetime and quantum yield suitable for monitoring hapten-antibody binding at nanomolar concentrations by fluorescence polarization wherein the concentration of said fluorophore tracer composition is in the range from about 1 mM to about 20 mM, the metal to tracer composition stoichiometry is about 1.0-1.1:1.0, and the pH of said acidic solution is about 2 or lower; and
b)adjusting the pH of the resulting solution to about 7 or greater.

9. A method for evaluating the metal selectivity of a macromolecular biological binding agent comprising:
A) combining serial dilutions of an aqueous solution thought to contain said biological binding agent with a fixed concentration of a first, target chelate-fluorophore tracer composition of claim 1, wherein M is the target metal, and measuring the polarization of the fluorescent signal obtained when each resulting solution is excited with plane-polarized light;
B) combining identical dilutions of said aqueous solution thought to contain the biological binding agent with a second, non-target chelate-fluorophore tracer composition of claim 1, wherein M is a non-target metal, said second tracer composition being present at the same concentration as the first tracer composition, and measuring the polarization of the fluorescent signal obtained when each resulting solution is excited with plane-polarized light;
C) subtracting the polarization signal produced by the solution containing the non-target tracer composition from that produced by the target tracer composition when measured at each sample dilution, whereby a positive net value at any dilution less than that producing a baseline signal for the target tracer composition indicates the presence of a macromolecular biological binding agent that binds selectively to the target chelate-fluorophore composition and a zero or negative net value indicates no selectivity for said target chelate-fluorophore composition; and
D) repeating steps B) and C) for as many non-target metals as may be required to fully define the metal selectivity of the macromolecular binding agent according to its intended purpose.

10. A method for evaluating the metal selectivity of a polyclonal antibody response in a target chelate-immunized animal comprising:
A) combining serial dilutions of serum drawn from said animal with a fixed concentration of a first, target chelate-fluorophore tracer composition of claim 1, wherein M is the target metal, and measuring the polarization of the fluorescent signal obtained when each resulting solution is excited with plane polarized light;
B) combining identical dilutions of said serum with a second, chelate-fluorophore tracer composition of claim 1, wherein M is a non-target metal, said second tracer composition being present at the same concentration as the first tracer composition, and measuring the polarization of the fluorescent signal obtained when each resulting solution is excited with plane polarized light;
C) subtracting the polarization signal produced by the solution containing the non-target tracer composition from that produced by the target tracer composition when measured at each sample dilution, whereby a positive net value at any dilution less than that producing a baseline signal for the target tracer composition indicates the presence of a polyclonal antibody that binds selectively to the target chelate-fluorophore composition; and
D) repeating steps B) and C) for as many non-target metals as may be required to fully define the metal selectivity of the polyclonal antibody according to its intended purpose.

11. A method for evaluating the metal selectivity of a monoclonal antibody present in a hybridoma supernatant or in a purified antibody preparation comprising:
A) combining serial dilutions of said hybridoma supernatant or purified antibody preparation with a fixed concentration of a first, target chelate-fluorophore tracer composition of claim 1, wherein M is the target metal, and measuring the polarization of the fluorescent signal obtained when each resulting solution is excited with plane polarized light;
B) combining identical dilutions of said hybridoma supernatant or purified antibody preparation with a second, chelate-fluorophore tracer composition of claim 1, wherein M is a non-target metal, said second tracer composition being present at the same concentration as the first tracer composition, and measuring the polarization of the fluorescent signal obtained when each resulting solution is excited with plane polarized light;
C) subtracting the polarization signal produced by the solution containing the non-target tracer composition from that produced by the target tracer composition when measured at each sample dilution, whereby a positive net value at any dilution less than that producing a baseline signal for the target tracer composition indicates the presence of a monoclonal antibody that binds selectively to the target chelate-fluorophore composition; and
D) repeating steps B) and C) for as many non-target metals as may be required to fully define the metal selectivity of the monoclonal antibody according to its intended purpose.

12. An immunoassay method for determining the concentration of a target metal ion in an aqueous solution comprising:
A) combining an aliquot of said solution with a first assay reagent comprising a buffered solution of EDTA, DTPA, or a derivative thereof;
B) adding to the resulting solution a second assay reagent comprising the corresponding target chelate-fluorophore tracer composition of claim 1, wherein M is the target metal;
C) adding to the second resulting solution a third assay reagent comprising a macromolecular biological binding agent that binds specifically to said target chelate-fluorophore tracer composition;
D) measuring the polarization of the fluorescent signal obtained when the third resulting solution is excited with plane-polarized light; and
E) comparing this value to those produced by standard solutions containing known concentrations of said target metal.

13. An immunoassay method for determining the concentration of a target metal ion in an aqueous solution comprising:
A) combining an aliquot of said solution with a first assay reagent comprising a buffered solution of EDTA, DTPA, or a derivative thereof and the corresponding target chelate-fluorophore tracer composition of claim 1, wherein M is the target metal;
B) adding to the resulting solution a second assay reagent comprising a macromolecular biological binding agent that binds specifically to said target chelate-fluorophore tracer composition;
C) measuring the polarization of the fluorescent signal obtained when the second resulting solution is excited with plane-polarized light; and
D) comparing this polarization value to those produced by standard solutions containing known concentrations of said target metal.

14. The immunoassay method of claim 12 or 13, wherein the aqueous solution is obtained by extraction of a solid sample, or a multiphasic sample that contains solids, with one or more aqueous mineral acids.

15. The immunoassay method of claim 12 or 13, wherein the aqueous solution is a water sample.

16. An immunoassay method for determining the concentration of lead in an aqueous extract of a solid sample, or of a multiphasic sample that contains solids, comprising:
A) combining an aliquot of said aqueous extract with a first assay reagent comprising a buffered solution of EDTA or a derivative thereof and the corresponding target chelate-fluorophore tracer composition of claim 3, wherein M is lead;
B) adding to the resulting solution a second assay reagent comprising a biological binding agent that binds specifically to said target chelate-fluorophore tracer composition;
C) measuring the polarization of the fluorescent signal obtained when the second resulting solution is excited with plane-polarized light; and
D) comparing this polarization value to those produced by standard solutions containing known concentrations of lead(II).

17. An immunoassay method for determining the concentration of lead in a water sample, comprising:
A) combining an aliquot of said water sample with a first assay reagent comprising a buffered solution of EDTA or a derivative thereof and the corresponding target chelate-fluorophore tracer composition of claim 3 wherein M is lead;
B) adding to the resulting solution a second assay reagent comprising a biological binding agent that binds specifically to said target chelate-fluorophore tracer composition;
C) measuring the polarization of the fluorescent signal obtained when the second resulting solution is excited with plane-polarized light; and
D) comparing this polarization value to those produced by standard solutions containing known concentrations of lead(II).

18. The immunoassay method of claim 16 or 17, further comprising an assay diluent comprising between about 10-100 mM sodium bicarbonate or HEPES, between about 10-100 μM EDTA, and between about 1-10 nM lead chelate-fluorophore tracer composition of claim 1 wherein M is Pb, n is 2, R₁ is p-CH₂C₆H₄—X—Y, R₂ is H; X is —HNC(S)NH—; and Y is fluorescein.

19. The immunoassay method of claim 16 or 17, further comprising an antibody comprising the rabbit polyclonal antiserum raised against the EDTA-Pb complex and screened for cross-reactivity with aluminum, iron, chromium, zinc, copper, and nickel.

20. A test kit for measuring the concentration of a target metal in a test sample, comprising:
A) at least one standard solution containing a known concentration of the target metal;
B) a first assay reagent comprising a base, a chelating agent, and the corresponding target metal chelate-fluorophore tracer composition of claim 1 wherein M is the target metal; and
C) a second assay reagent containing a known concentration of the biological binding agent responsive to the target metal chelate-fluorophore tracer composition.

21. A test kit for measuring the concentration of lead in a test sample, comprising:
A) at least one standard solution containing a known concentration of lead(II);
B) a first assay reagent comprising a base, EDTA, and the corresponding lead chelate-fluorophore tracer composition of claim 3 wherein n is 1, Y is fluorescein, and M is Pb;
C) a second assay reagent containing a known concentration of the biological binding agent responsive to the lead chelate-fluorophore tracer composition; and
D) optionally, an extraction fluid suitable for obtaining an aqueous extract of a solid sample, or a multiphasic sample that contains solids.
